(19) United States
(12) Patent Application Publication
(10) Pub. No.: US 2009/0123487 A1
(43) Pub. Date: May 14, 2009

(54) Precursors and enzymes associated with post translational modification of proteins implicated in isoform generation of PCNA

(76) Inventor: Katia Rothhaar, Reelsville, IN (US)

Correspondence Address: WHITEFORD, TAYLOR & PRESTON, LLP;ATTN: GREGORY M STONE, SEVEN SAINT PAUL STREET, BALTIMORE, MD, 21202-1626, US

(21) Appl. No.: 12/284,241

(22) Filed: Sep. 19, 2008

Related U.S. Application Data
(60) Provisional application No. 60/994,417, filed on Sep. 19, 2007.

(51) Int. Cl. A61K 39/00; C12Q 1/02; C12Q 1/68; C07H 21/04; C07H 21/02; C07K 16/18

(52) U.S. Cl. 424/184.1; 435/29; 435/6; 536/24.31; 530/387.3

(57) ABSTRACT

The current invention provides a method for detecting the presence of a genomic or proteomic precursor(s) within a sample, wherein the precursor(s) provide an indication of the presence or the capability of expression modulation of various other proteins which may, either directly or indirectly, provide an indication, promote, and/or be responsible for the post translational modification of the PCNA.

### CROSS REFERENCE TO RELATED APPLICATION

The present application claims priority under 35 U.S.C. §119 to the U.S. Provisional Patent Application Ser. No. 60/994,417, filed on Sep. 19, 2007 which is herein incorporated by reference in its entirety.

### FIELD OF THE INVENTION

The present invention generally relates to the field of oncology, and particularly to the detection and use of proteins (enzymes) that may promote and/or be responsible for the translational modifications of proteins, such as Proliferating Cell Nuclear Antigen and others, involved in the DNA replication process.

### BACKGROUND OF THE INVENTION

Revolutionary strides in the field of cancer have been made over the last several decades. Advances in treatments from chemotherapies, to irradiative treatment, to refining the procedures through which cancerous tissues are removed, have provided everything from cures to some, to extended life to others. The early diagnosis of cancer has remained a difficult problem due in large part to the complexity of the disease process as cells undergo the transformation from normal to malignant. The DNA replication mechanism is often a leading indicator of malignancy, whereby noted abnormalities of this process, such as over/under synthesis, improper timing, inconsistent replication fidelity, can provide key indicators that a problem exists. Today, advances in detection methodologies have had a significant impact upon the prevention of cancer-related deaths. Detection methodologies such as improved X-ray and CAT scan techniques may allow for the detection of cancer well before it is visible or palpable. Other techniques, such as the use of antibodies with high affinity and selectivity to antigens expressed in malignant cells have also proven to be useful tools. Still today, however, it is often the case that cancer diagnosis is unable to reliably occur until well beyond the early stages of cell transformation and potentially long after a cell's pre-cancerous phase.

Typically, the detection techniques employed, even advanced ones such as those mentioned above, are simply a first step in the diagnosis process of the diseased cells. The early detection techniques may provide only a general indication of the possible presence of a malignancy, lacking the ability to provide reliable and specific deterministic results. Thus, the early detection techniques may require follow up procedures, such as biopsy of the malignant cells and histological examination, to make a reliable determination whether a tissue is malignant or benign. Where a tissue is finally found malignant it may be determined that a resection of the afflicted tissue is warranted in order to determine the progression of the disease and provide a reliable prognosis. However, resection may commonly result in the removal of both malignant and benign growths. Therefore, such an invasive technique may only provide limited effectiveness and may result in serious harm to the patient, extended recovery periods, and lengthy hospital stays. This systematic approach may also be quite time consuming and in the filed of oncology the time at which a cancer diagnosis is made can be quite critical, possibly the difference between the ability to successfully treat the disease and only being able to manage the disease's progression. Current cancer therapeutics are not specific for cancer versus normal (non-malignant) cells and often lead to many diverse side effects.

While cancers are unique, it has been discovered that malignant cells share some common attributes. (see U.S. Pat. No. 6,063,575; U.S. Pat. No. 6,093,543, which are herein incorporated by reference in their entireties). Thus, while the process of malignancy involves both genetic and proteomic transformation, it may be possible to identify one or more of the common attributes. Unfortunately, the identification of these attributes as biomarkers specific for malignancy occurrence and progression has proven difficult. Currently employed techniques designed to identify such biomarkers often provide a generic identification of a biomarkers presence, but are unable to provide a clear delineation between the malignant and normal cellular state and/or provide clear indication regarding the progression of the disease. Therefore, the results achieved through use of known, commercially available markers and techniques are not specific for malignancy assessment and require further examination before useful information is developed. Further, the identification of many biomarkers for cancer using the current techniques is only able to occur after the malignancy has been identified in a patient, typically due to the biomarker reaching a sufficiently elevated level of presence in the patient such that these techniques are enabled to detect its presence. Unfortunately, this often results in a situation where the current techniques are useful only after the malignancy has been identified in a patient, thereby allowing for treatment but not able to enable/promote preventative assistance.

### SUMMARY OF THE INVENTION

The present invention provides a method for detecting qualitative and quantitative indicators, such as proteins and/or other precursors, which may be associated with or assist in accomplishing the post translational modification of isoforms that are part of the DNA replication process which may, either directly or indirectly, provide an indication of a malignancy or be involved in promoting, assisting with, associating with and/or being responsible for a malignancy. Also, it provides the ability to identify, characterize and measure new markers for such proteins and/or precursors which may indicate a malignancy or predisposition towards malignancy. Further, the current invention provides a rapid, minimally invasive technique for diagnosing, prognosing and monitoring a disease and/or therapeutic response. It also provides therapeutics and vaccines that are able to specifically target the diseased cells and have limited to no impact upon non-diseased cells.

The present invention contemplates various exemplary approaches to providing a novel detection system(s) and method(s) for reliably detecting and or monitoring the existence of malignancy and/or the potential for malignancy within various samples, such as cells, tissue, serum, and the like. In an exemplary embodiment of the current invention, a method for detecting and quantitatively measuring the presence of a genomic precursor form (i.e., DNA, cDNA, RNA (mRNA)) that may promote, assist, be associated with and/or responsible for the modification of PCNA, where modification may occur at various stages of the proteins life cycle and through various mechanisms (e.g., methylation), into the isoform cancer associated PCNA (caPCNA). In another exemplary embodiment, a method for detecting the presence of a proteomic precursor (i.e., proteins and/or enzymes) that may promote, assist, be associated with and/or responsible for modifying caPCNA into a normal (non-malignant) PCNA (nPCNA or nmPCNA) isoform is provided.

In a contemplated exemplary embodiment of the current invention, a biomarker or high affinity agent is provided that allows for the qualitative and/or quantitative detection of a genomic or proteomic precursor that may promote, assist, be associated with or be responsible for the post translational modification of the PCNA isoform. Thus, the biomarker may allow for a malignant or potentially malignant aspect of a tissue to be detected.

In a contemplated exemplary embodiment of the current invention a method for quantifying the presence of a precursor in a sample is provided. In another contemplated exemplary embodiment of the current invention, a screening system and method are provided. The screening methodology may be provided for application within a kit or for use within a laboratory environment. It is further contemplated that the current invention provides a quantification system and method. This capability may be provided as part of the screening system or method or as a separate feature, such as a kit or laboratory based system and methodology.

In another contemplated exemplary embodiment of the current invention, a diagnostic method and kit is provided whereby the current invention allows for a reliable determination of the presence of the malignancy. In another contemplated exemplary embodiment, a prognostic method is provided whereby the current invention is able to determine the stage/progression of the disease and assist in the prediction of future growth and disease progression. In still another contemplated exemplary embodiment, a method for monitoring the progression of a disease and/or therapeutic incidence on the disease state is provided by the current invention.

In another contemplated exemplary embodiment of the current invention a therapeutic is provided that may allow for the treatment of the disease. The therapeutic may be able to target the malignant cells and/or those that have pre-malignant indicators. The therapeutic may be combined with various other therapeutics as may be contemplated by those of ordinary skill in the art. In another exemplary embodiment, the current invention may provide a vaccine.

It is to be understood that both the foregoing general description and the following detailed description are exemplary and explanatory only and are not restrictive of the invention as claimed. The accompanying drawings, which are incorporated in and constitute a part of the specification, illustrate an embodiment of the invention and together with the general description, serve to explain the principles of the invention.

### BRIEF DESCRIPTION OF THE DRAWINGS

The numerous advantages of the present invention may be better understood by those skilled in the art by reference to the accompanying figures in which:

FIG. 1 is a block diagram illustration of a method of detecting a precursor in accordance with an exemplary embodiment of the present invention.

FIG. 2 is a block diagram illustration of a method of diagnosing a disease by detecting a precursor in accordance with an exemplary embodiment of the present invention.

### DETAILED DESCRIPTION OF THE INVENTION

Reference will now be made in detail to the presently preferred embodiments of the invention, examples of which are illustrated in the accompanying drawings.

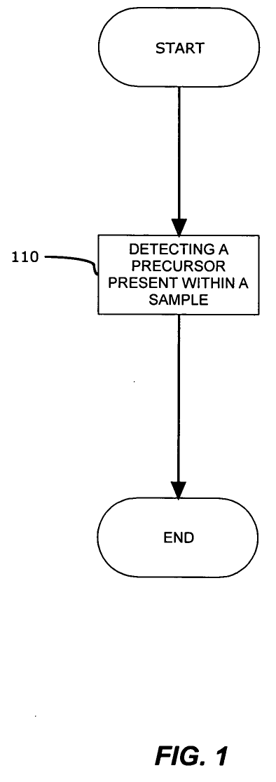

In a preferred embodiment, a method for detecting genomic and proteomic “precursors” is shown in FIG. 1. The precursors may exist within an intracellular, intercellular or extracellular environment, and provide an early stage indicator of the capability to produce certain proteins (e.g., enzymes) or indicate the presence of these proteins that may, either directly or indirectly, promote and/or be responsible for the post translational modification of other proteins, such as Proliferating Cell Nuclear Antigen (PCNA), which play a role in DNA replication.

Genomic precursors referred to herein may include DNA, cDNA, and/or RNA (mRNA) templates for these enzymes that may be associated with the post translational modification of the PCNA isoform. Proteomic precursors may include various proteins/enzymes such as methyltransferase.

The isoforms of PCNA will be referred to herein as normal PCNA (nPCNA) and cancer associated PCNA (caPCNA). The nPCNA is found in healthy non-malignant cells and the caPCNA is found in malignant cells and/or cells that are in the process of becoming malignant. Typically, the isoforms of PCNA may be found in the intracellular space and take part in the DNA synthesome complex. The DNA synthesome complex provides the mechanism through which DNA synthesis/replication activity takes place. Thus, PCNA is an element of the DNA synthesis/replication mechanism.

For the genomic precursors, the templates detected by this method may be various precursor forms, such as fragments, or other designations of varying sequence length. It is further contemplated that various hybridized forms of the precursors may be detected by the present invention such as siRNA. These precursor forms may be a part of the process through with the synthesis of various different enzymes may occur or may be expressed by the proteins/enzymes in its various isoforms.

The method 100 (FIG. 1) may include the step 110 of detecting a precursor present within a sample. The sample may be from various sources, such as tissue or body fluid, however, the source of the tissue or body fluid is not critical for the current invention. For example, tissue employed as the sample for the current invention may be cervical, mammary glands, esophageal, prostate, lung, stomach, intestine, glial cells or white blood cells. Examples of various body fluids that may be utilized may include saliva, urine, serum, whole blood and the like.

While the detection of precursors, that may include various genomic indicators and/or proteins (enzymes), present within the sample that may be responsible for the post translational modification of the PCNA isoform may require a sample from a cancer afflicted source, the particular type of cancer or the stage of the disease is not critical to the current invention. Therefore, a sample may be obtained from a source afflicted with carcinomas, sarcomas, lymphomas, or leukemias. Examples of such cancers include cervical carcinoma, mammary gland carcinoma of ductal or lobular origin, gliomas, prostate, lung, esophageal, stomach and ovarian cancer. It is contemplated that the sample may include various tissues/body fluids at various stages of cancer development, for example pre-cancerous tissues/body fluids or tissues/body fluids at various other early stages of the development of a malignancy may be utilized by the present invention. The various tissues and body fluids that may be utilized by the current invention may preferably include the precursors which are responsible for the post translational modification of the PCNA isoform. It is further contemplated that the sample may be from a source that is not currently exhibiting a malignant/cancer phenotype, but instead may be at a pre-cancerous stage.

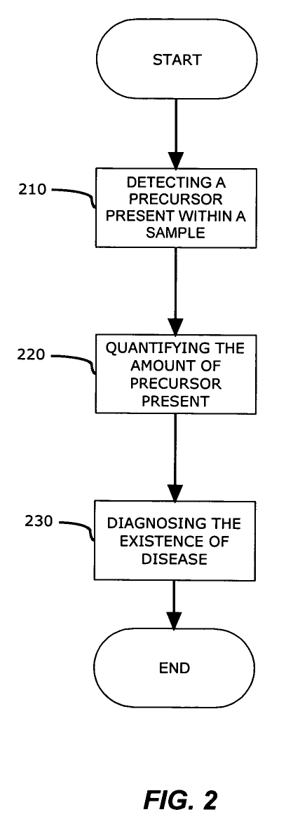

In general, the detection of the precursors in a sample may also allow for the diagnosis of a disease within a biological system (FIG. 2). For instance, the identification/quantification of the amounts of the precursor forms present in a sample may allow for the ascertainment of what precursor forms are present and in what concentration. From this determinations may be made as to whether the precursor(s) may be synthesized or what the expression ratios within the sample are and then allow for the determination of which of those proteins may be significant for the post translational modification of the PCNA isoform. This analysis may lead to the diagnosis of the presence of a disease within a biological system at various stages of the disease. Most significantly, the current invention may allow for the diagnosis of the presence of a disease at a very early stage, such as a pre-cancerous stage, in the onset of a disease. This may further promote the effectiveness of various treatments, including the use of various therapeutics in conjunction with the affinity agent that is used to detect the presence of the precursors as is discussed below.

The current invention provides a method for detecting the precursors, including the proteomic precursors or proteins, associated with the post-translational modifications of isoforms of Proliferating Cell Nuclear Antigen (PCNA). Thus, the method for detecting the presence of a precursor, as shown in FIG. 1, may further refine the detection step to that of detecting one or more proteins, which may be responsible for or be active in promoting the post translational modification of the PCNA isoform present within a sample. In a preferred embodiment, the protein detected is a methyltransferase which has been identified as at least a part of the post translational modification of PCNA. Other proteins (associated proteins or enzymes) which accomplish or assist in accomplishing post translational modifications of other proteins, as are known by those skilled in the art, may also be detected by the current invention.

The caPCNA isoform has been discovered to include a methylation, as a result of a post translational modification process which includes methyltransferase, which is not found within the nPCNA. Thus, it is contemplated that the method of the current invention may allow for the detection of proteomic precursors/enzymes that both add and remove the methyl from the PCNA isoforms.

It is contemplated that the method for detecting the protein(s) may also include a step of obtaining a “biomarker” or high affinity agent (e.g. a detector) and utilizing the detector to bind with and detect the presence of the protein. For example, a biomarker or high affinity agent may include a complementary sequence to an epitope located on the target (i.e., methyltransferase). As will be described below, the detector may be constructed in various configurations, such as an antibody that may be constructed to include the complementary sequence and bind to and/or with the target.

In a still further step an indicator or “label” may be included with the biomarker or high affinity agent/complementary sequence to allow for the detection of the binding of the detector to the precursor target. As will be described herein below, the label may be of various design, such as radioactive, fluorescent, other immunochemical label, and the like as may be contemplated by those of ordinary skill in the art. It is further contemplated that the detector may work alone or in combination with various other secondary antibodies, proteins, reagents, delivery mechanisms (i.e., liposomal), and the like. Thus, the detection of the precursor may include the step of detection through use of a simple assay technique or one or more alternative techniques, such as a secondary antibody detection technique, which are well known in the art. One could design an antibody specific to a proteomic precursor which could be used in ELISA type bioassay with a HRP labeled type polymer conjugated with secondary antibodies

The current invention is capable of detecting the precursors forms which may assist, promote, or provide for the reciprocal conversion of the PCNA isoform. Thus, the detection of the precursor forms may allow for the detection of bi-directional post translational modification. For example, a first precursor may be detected that is responsible for, assists in and/or promotes in some manner the conversion of nPCNA to caPCNA. The current invention may also detect a second precursor that is responsible for, assists in and/or promotes in some manner the conversion of caPCNA to nPCNA. It is contemplated that a single precursor may assist, promote, or be responsible for the “reciprocal conversion” of the PCNA isoform. Table 1, below, illustrates the serial changes of expression of the PCNA isoform that the precursor(s) which may be detected by the current invention may be responsible for.

TABLE 1
Enzyme Mediated Reciprocal Conversion

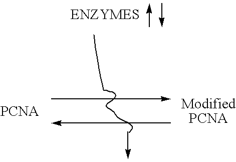

The previous embodiments of the current invention have identified a detection method for a precursor that may assist, promote, or be responsible for the post translational modification of PCNA. It is contemplated that the current invention may detect these precursors in conjunction with one or more other proteins, such as, oncoproteins, angiogenic factors, tumor markers, inhibitors, growth factor receptors, metastasis proteins, and tumor suppressors.

The techniques which may be employed by the current invention for detection of the proteomic and/or genomic expression of these post translational modifiers of the PCNA isoform may vary as contemplated by those of ordinary skill in the art. For example, real-time quantitative PCR or any other type of PCR assays and/or immunoassay type assays may be employed. These and other techniques may allow for the detection, amplification and quantification of proteomic and/or genomic expression proteins and/or the precursors associated with the post translational modification of the PCNA isoform. Additional detections may include mass spectrometry, Raman spectroscopy, SERS, SERRS, fluorescence and chemiluminescence.

The current invention may include a method of developing a technique, such as an assay or PCR technique, that allows for the detection, amplification and quantification of proteomic and/or genomic expression of indirect or direct participants and/or precursors associated with the post translational modification of the PCNA isoform.

It is contemplated that the various embodiments of the current invention described herein may employ various secondary features to affect the processes taking place in the detection, amplification, and quantification of the proteins and/or precursors that may be responsible for the post translational modification of the PCNA isoform. For example, various catalysts, delivery mechanisms and the like which may increase activity or various features that decrease activity may be employed.

In a preferred embodiment, the present invention provides a method for quantifying the amount of protein and/or precursor present within the sample. The method includes the step of quantifying the results of the detection of the presence of a precursor and/or protein within a sample. It is contemplated that this quantification step may be employed in conjunction with the alternative exemplary methods described herein. This quantification step may be enabled through various methodologies, such as various labeling techniques (i.e., radioactive, fluorescence) and the like which may allow for a visual determination and/or other measurable determinant as contemplated. The quantification may be directed to the concentration of the protein and/or precursor present within the sample. The quantification may be further refined to allow for a determination of a specific amount of the protein and/or precursor which is present within the sample.

Thus, the present invention provides a method for determining an amount of precursor present within a sample. The current invention may provide a method for determining a relationship between the amount of precursor present within a sample and the association, if any, of the amount present with the conversion (reciprocal or not) of the PCNA isoform. The method may include the steps of determining an amount of the precursor present within a sample, determining the amount or rate of conversion of a PCNA isoform within the sample, a ratio between the amount of precursor present and the converted PCNA isoform. Thus, the method may allow for the determination of a relationship between the amounts (e.g., starting amount) of the precursor found in a sample and the likely amount of modified PCNA already present and/or likelihood of the sample exhibiting a modified PCNA isoform. From this the current invention, as described herein, may allow a person skilled in the art to make a determination as to the stage of malignant development that may occur, is occurring or has occurred within the sample. The determination of what precursors and in what amounts may cause and/or lead to the modification of the PCNA isoform whether it is from nPCNA to caPCNA or the reciprocal conversion may also allow for the prognosis and monitoring of the stage of development of the disease/malignancy.

In a preferred embodiment, a biomarker may be provided by the current invention for the detection/identification of a precursor form that may be assisting, promoting or responsible for the post translational modification of PCNA isoform. It is contemplated that the biomarker of the current invention may be a ligand (nucleotide sequence) that may hybridize with or be hybridized to a complementary sequence that has a high binding affinity for a particular sequence that is located within the precursor found within the sample and is indicative of the expression of a malignancy or potential malignancy indicator. It is also contemplated that the biomarker may be configured with a high binding affinity for a particular region on a protein (i.e., epitope) isoform that is expressed within a malignant or potentially malignant sample. Various exemplary embodiments of a genomic and/or proteomic biomarker may be provided as a small molecule, siRNA, antibody or other commonly known and used structural and delivery configurations.

The current invention may provide a method for providing a biomarker capable of detecting a precursor that may be responsible for the post translational modification of the PCNA isoform. The providing of the biomarker may include a first step of identifying a target and a second step of constructing a high affinity agent including a high affinity region that may allow for binding and/or hybridization with the target. The high affinity agent, which may also serve the function of delivering the high affinity region, may be of various forms, such as an antibody, small molecule, and the like as may be contemplated by those of skill in the art. The high affinity region may be provided as a ligand or other sequence structure that may be specific for the target. It is further contemplated that the biomarker may be used in conjunction with various secondary features, such as secondary antibodies, liposomes, labels, reagents, catalysts, activators, inhibitors, and the like as have been discussed previously and are well known in the art.

It is contemplated that the biomarkers provided by the current invention, such as the biomarker constructed in antibody form may be able to detect the presence of precursors in various samples which may be pre-cancerous through various other stages of the disease progression. For example, the biomarkers of the current invention may detect precursors in pre-malignant cells or malignant cells in tissues such as breast, prostate, colon, rectal, skin (epithelial and endothelial), esophageal, liver, cerebral, pancreas, stomach, testicles, ovaries, heart, bladder, kidney, intestine, blood, muscle (smooth or striated), spleen, cervix, adrenal gland, pituitary gland, lymph node, lungs, or in various cell lines.

The detection by the biomarkers of the precursors may be accomplished through hybridization/binding with cells that may be part of a malignant tumor or with cells that have broken free from the tumor and are present within the blood of the patient. It is to be understood that the current invention may be employed with patients of various species of the mammalian genus. For example, it is contemplated that the patient may be a human mammal or another species, such as apes.

In another preferred embodiment, a method of screening a sample for the presence of a precursor that may be responsible for the post translational modification of PCNA isoform is provided. In a still further embodiment, a system for screening a sample for the presence of a precursor that may be responsible for the post translational modification of PCNA isoform is provided. The screening methodology and system may be provided for application within a kit or for use within a laboratory environment. It is further contemplated that the quantification capability previously described above may be provided as part of the screening system or method or as a separate feature.

The screening method may include the step of obtaining a sample. The sample may or may not be suspected of having the presence of a precursor of or malignancy. The sample may then be included within a reaction mixture. The reaction mixture may include one or more known and labeled biomarkers that have a high affinity for a known precursor that may be responsible for the post translational modification of a PCNA isoform. It is contemplated that the known precursor assessment that may be responsible for the post translational modification of a PCNA isoform could be combined with the detection of additional biomarkers such as Ki-67, PSA, CA-125, and the like. The method may further include the step of detecting the bound biomarker/protein and/or precursor complex. The detection may occur utilizing the assay or real-time PCR techniques identified previously or other assaying techniques known and used by those of skill in the art.

The screening system may be a kit including a sample obtaining component, a reaction mixture, and a detection technique. The kit may further include components, such as labeling reagents, buffers, catalysts, other reagents, and the like. The detection technique may include the capability to perform an immunochemical staining, RIA, ELISA, and/or other assay techniques. The sample obtaining component may be of general design for collecting tissue or body fluid as is well known by those skilled in the art. The reaction mixture may include one or more biomarkers for various precursors that may be responsible for the post translational modification of the PCNA isoform. The kit may further include instructions for its use and various other tools which may be employed in performing the screening method as described previously. It is contemplated that a computer aided analysis of the results may be of general design for assessing the sample results.

The current invention provides a diagnostic method 200 (FIG. 2) for the presence of a malignancy or a potential malignancy. The diagnostic method may include a first step 210 of detecting the presence of a precursor in a sample from a subject or patient. The presence of the precursor may then be quantified in a second step 220. The quantification may then allow for the diagnosis of the absence or presence of a malignancy or potential malignancy within the sample in a third step 230. It is contemplated that the diagnostic capability of the present invention may allow for diagnosis of pre-cancerous to later stage disease progressions. The diagnostic method may further include the step of obtaining a sample from a patient. The diagnostic method may further include the combination and/or ratio of the precursor with normal PCNA and/or with known biomarkers such as Ki-67, PSA, CA-125, and the like. It is contemplated too that the presence of a precursor could be used as a target for imaging tool such as a PET ligand.

It is contemplated that the current invention may include a diagnostic kit that allows for the performance of the diagnostic method previously described. The diagnostic kit may include many similar components as that described above for the screening kit. The diagnostic kit may further include tools, software and components for performing the quantification step of the diagnostic method.

The various diseases that may be diagnosed utilizing the methods, systems, and kits of the current invention may include all cancers, such as melanomas, leukemias, lymphomas (lymphocitic, myelogenous, Hodgkins, non-Hodgkins), hepatomas, nephromas, astrocytomas, carcinomas (i.e., breast, bladder, prostate, esophageal, ovarian, adrenal (adrenocortical), cervical, lung), blastomas (i.e., glio-, lympho-, retino-, neuron-), endometrial, gliomas, sarcomas (i.e., uterine, osteo), and many other variants of the cancerous forms.

It is further contemplated, in another exemplary embodiment, that the current invention provide a prognostic method whereby the current invention is able to assist in the determination of the stage/progression of the disease and assist in the prediction of future growth and disease progression. The prognostic capability may be enabled through the quantification analysis of the current invention. The determination of certain amounts and/or percentages of the precursor(s) and/or combination with additional biomarkers present within the sample may allow the identification of disease progression and prediction of future growth. The prognostic method may include the steps of quantifying the presence of the precursors at a non-cancerous, pre-cancerous stage or early stage of the disease and then correlating the level of presence of those precursors at various later stages of the disease progression.

In still another contemplated exemplary embodiment, a method for monitoring a disease is provided by the current invention. In a first step a first sample is taken and the presence of the precursor and/or combination with additional biomarkers is detected and quantified. In a second step, after the expiration of some time period from the performance of the first step, a second sample is taken and the presence of the precursor and/or combination with additional biomarkers is detected and quantified. In another step, the quantification from the first sample is compared against the quantification from the second sample. It is contemplated that while this is an exemplary monitoring method, these steps and others may be employed for purposes of accomplishing the prognostic method described above. It is further contemplated that this method of monitoring may be employed as part of a method of monitoring how a patient is responding to a therapy (i.e., treatment regime), and drug development screening. It is contemplated that this method will allow earlier detection of cancer recurrences.

A composition of matter is provided that may allow for the treatment of a disease as identified previously. In a preferred embodiment, a composition of matter may be a therapeutic containing a high affinity agent and a therapeutic agent. The high affinity agent may be able to specifically target a diseased cell and/or those that have pre-disease onset indicators by targeting the precursors that may assist, promote, and/or be responsible for the post translational modifications of the PCNA isoform. Once hybridized/bound with the precursor the therapeutic agent is enabled to take effect. It is contemplated that the high affinity agent may be combined with various therapeutic agents as may be contemplated by those of ordinary skill in the art.

In operation, the therapeutic agent of the composition of matter may compete for the binding of the precursor at the sites where these precursors bind with various other proteins which may then be activated to perform or assist in the performance of the post translational modifications on the PCNA isoform which may lead to or be associated with the malignant development of the cell and tissue. The composition of matter may include a therapeutic agent that effectively renders a precursor(s) inactive. It is also contemplated that the composition of matter may promote, assist or be responsible for the removal of a precursor(s) from a sample or a biological system, such as a mammalian biological system.

It is further contemplated that the composition of matter of the current invention may be formulated into a vaccine that allows for the delivery of staged disease samples to a recipient and assist or promote the ability of the recipient to develop or provide an immune response that is capable of targeting the expressed proteomic and/or genomic indicators of the disease.

In combination with therapeutic or vaccine formulations other products, such as other secondary or supplemental components may be utilized. For instance, activators may be used to increase enzymatic activity or inhibitors may be used to decrease enzymatic activity. Further, the therapeutic, particularly in antibody form, may be bound to and delivered utilizing liposomes or other carriers which may allow the therapeutic to be delivered to an intra- or inter-cellular space for targeting a specific precursor. It is contemplated that the therapeutic may be delivered in various forms, such as small molecule and the like. For example, ligands of the PCNA isoform post translational modifications enzymes may be incorporated into such small molecules as therapeutics for preneoplastic/neoplastic diseases. Such ligands could be activator and/or suppressor of PCNA isoforms post translational modification enzymes in combination with or independent of additional therapies. The therapeutics and/or vaccines may include antibodies such as those described previously for providing biomarkers that bind with the precursors. The antibodies may be full length antibodies, fragments, or antibody fusion proteins as is known in the art.

The administration of the various therapeutic and/or vaccine formulations may vary. Preferably, administration may occur parenterally (injection, intravenous), orally, rectally, topically, and various other mechanisms as contemplated by those of skill in the art. The amounts (i.e., dosage) of therapeutic and/or vaccine may be dependent on many factors, such as the level of presence of the protein and/or precursor (stage of disease progression), composition of the therapeutic/vaccine itself, the condition of the patient and other factors.

In the exemplary embodiments, it is to be understood that the specific order or hierarchy of steps in the methods disclosed are examples of exemplary approaches. Based upon design preferences, it is understood that the specific order or hierarchy of steps in the method can be rearranged while remaining within the scope and spirit of the present invention. The presentation of the various steps in a sample order are not necessarily meant to be limited to the specific order or hierarchy presented.

It is believed that the present invention and many of its attendant advantages will be understood by the forgoing description. It is also believed that it will be apparent that various changes may be made in the form, construction and arrangement of the components thereof without departing from the scope and spirit of the invention or without sacrificing all of its material advantages. The form herein before described being merely an explanatory embodiment thereof.

What is claimed is:

1. A method of identifying/detecting an oncological post-translational agent, comprising:
detecting the presence of a precursor within a sample.

2. The method of claim, further comprising quantifying the presence of the precursor.

3. The method of claim, further comprising at least one of diagnosing, prognosing or monitoring the presence of a disease based on the presence of the precursor.

4. The method of claim, wherein the disease is at least one of carcinomas, sarcomas, hepatomas, nephromas, astrocytomas, melanomas, gliomas, blastomas, endometrial, lymphomas, or leukemia.

5. The method of claim, wherein the sample is at least one of a tissue, body fluid, or cell and the source of the tissue is at least one of cervical, mammary glands, prostate, lung, stomach, intestine, glial cells or white blood cells, breast, colon, rectal, skin (epithelial and endothelial), esophageal, liver, cerebral, pancreas, testicles, ovaries, heart, bladder, kidney, intestine, blood, muscle (smooth or striated), spleen, cervix, adrenal gland, pituitary gland, or lymph node.

6. The method of claim, wherein the source of the body fluid is at least one of saliva, urine, serum, or whole blood.

7. The method of claim, wherein the cells are at least one of pre-malignant cells or malignant cells in tissues. such as

8. The method of claim, further comprising employing at least one of PCR, mass spectrometry, Raman spectroscopy, SERS, SERRS, fluorescence or chemiluminescence techniques for the detection of the precursor.

9. A biomarker, comprising:
a high affinity agent capable of detecting a precursor.

10. The biomarker of claim, wherein the precursor is at least one of a genomic or proteomic precursor.

11. The biomarker of claim, wherein the genomic precursor is at least one of DNA, cDNA, RNA or mRNA.

12. The biomarker of claim, wherein the proteomic precursor is at least one of methyltransferase

13. The biomarker of claim, wherein the precursor is found in conjunction with at least one of oncoproteins, angiogenic factors, tumor markers, inhibitors, growth factor receptors, metastasis proteins, or tumor suppressors.

14. The biomarker of claim, wherein the affinity agent includes a high affinity region that provides specificity for a target included on the precursor.

15. The biomarker of claim, wherein the affinity agent is at least one of a small molecule, siRNA, antibody, antibody fragment, antibody fusion protein.

16. The biomarker of claim, wherein the affinity agent includes an indicator selected from the group consisting of radioactive, immunochemical, and fluorescent labels.

17. The biomarker of claim, wherein the affinity agent includes a secondary feature selected from the group consisting of secondary antibodies, liposomes, labels, reagents, catalysts, activators, and inhibitors.

18. A composition of matter, comprising:
a high affinity agent and a therapeutic agent, wherein the therapeutic agent interacts with the precursor upon targeted delivery by the affinity agent.

19. The composition of claim, wherein the composition may be formulated in at least one of a solid, liquid, or gas phase.

20. The composition of claim, wherein the composition is a vaccine.
